(19) United States
(12) Patent Application Publication
(10) Pub. No.: US 2022/0318613 A1
(43) Pub. Date: Oct. 6, 2022

(54) DEEP LEARNING MODELS AND RELATED SYSTEMS AND METHODS FOR IMPLEMENTATION THEREOF

(71) Applicant: Express Scripts Strategic Development, Inc., St. Louis, MO (US)

(72) Inventors: Balakrishnan Nambirajan, Parsippany, NJ (US); Matthew Cole, University City, MO (US)

(21) Appl. No.: 17/219,955

(22) Filed: Apr. 1, 2021

(51) Int. Cl. G06N 3/08; G06N 3/04; G06F 16/28

(52) U.S. Cl. CPC G06N 3/08; G06N 3/0445; G06N 3/0427; G06F 16/283

(57) ABSTRACT

A machine learning system is provided for training a data model to predict data states. The machine learning server is configured to receive a first portion of historical pharmaceutical data. The machine learning server is configured to apply a deep learning variable importance method to the first portion to identify at least one salient variable. The machine learning server is also configured to apply the model generation algorithm to the first portion and the at least one salient variable to generate predictive models for the forecast of the target variable. The machine learning server is also configured to receive a second portion of the historical pharmaceutical data to test the predictive models. The machine learning server is also configured to obtain a portion of current pharmaceutical data and apply the portion of current pharmaceutical data to the candidate predictive model to obtain the forecast of the target variable.

### FIELD

The field relates to deep learning models and related machine learning systems and methods implementing such models. The deep learning models described are used to predict trends in data including pharmaceutical claims data and to forecast price changes in markets including prescription drug markets.

### BACKGROUND

Rendering accurate determinations about trends in data sets is an often crucial in pharmaceutical claims processing and management. However, known methods of forecasting are highly error prone and otherwise unreliable. In one aspect, known methods depend heavily on human manual input and are therefore prone to errors when information is imprecisely captured or entered. Further, it is difficult or impossible for known automated systems to address the problems of predicting trends such as price trends in pharmaceutical claims data. This is because known methods presume that static data models are used to create trend predictions. In reality, underlying conditions in trends evolve constantly and in unpredictable manners. As such, known automated methods fail to address the underlying uncertainty in models for predicting trends.

Therefore, in existing pharmaceutical claims processing systems, static otherwise limited data models are used, causing the systems to erroneous and improper results predictions. In many examples, systems can only be improved through the use of manual verification steps, errors, or intermittent manual updates to models. Even with such improvements, the risk of erroneous and improper forecast data remains.

As such, deep learning models described are desired in order to predict trends in data including pharmaceutical claims data and to forecast price changes in markets including prescription drug markets.

### BRIEF SUMMARY

In one aspect, a machine learning system is provided for training a data model to predict data states. The machine learning system includes a first data warehouse system and further includes a warehouse processor and a warehouse memory. The first data warehouse system further includes historical pharmaceutical data associated with one or more pharmaceuticals. The first data warehouse system also includes a machine learning server in communication with the first data warehouse system. The machine learning server includes a processor and a memory. The machine learning server is configured to receive a first portion of historical pharmaceutical data. The first portion includes variables associated with a forecast of a target variable. The machine learning server is configured to apply a deep learning variable importance method to the first portion to identify at least one salient variable. The machine learning server is also configured to apply a combination of a long-short term memory algorithm, a multilayer perceptron algorithm, and a predictive artificial intelligence algorithm to generate a model generation algorithm. The machine learning server is also configured to apply the model generation algorithm to the first portion and the at least one salient variable to generate predictive models for the forecast of the target variable. The machine learning server is also configured to receive a second portion of the historical pharmaceutical data to test the predictive models. The machine learning server is also configured to test the predictive models with the second portion to identify a candidate predictive model that most accurately forecasts the target variable based on the second portion. The machine learning server is also configured to obtain a portion of current pharmaceutical data and apply the portion of current pharmaceutical data to the candidate predictive model to obtain the forecast of the target variable.

In another aspect, a method is provided for training a data model to predict data states. The method is performed by a machine learning system including a machine learning server and further including a first data warehouse system. The first data warehouse system further includes a warehouse processor and a warehouse memory. The first data warehouse system further includes historical pharmaceutical data associated with one or more pharmaceuticals. The first data warehouse system also includes the machine learning server that is in communication with the first data warehouse system. The machine learning server includes a processor and a memory. The method includes receiving a first portion of historical pharmaceutical data. The first portion includes variables associated with a forecast of a target variable. The method also includes applying a deep learning variable importance method to the first portion to identify at least one salient variable. The method further includes applying a combination of a long-short term memory algorithm, a multilayer perceptron algorithm, and a predictive artificial intelligence algorithm to generate a model generation algorithm. The method further includes applying the model generation algorithm to the first portion and the at least one salient variable to generate predictive models for the forecast of the target variable. The method also includes receiving a second portion of the historical pharmaceutical data to test the predictive models. The method additionally includes testing the predictive models with the second portion to identify a candidate predictive model that most accurately forecasts the target variable based on the second portion. The method also includes obtaining a portion of current pharmaceutical data and applying the portion of current pharmaceutical data to the candidate predictive model to obtain the forecast of the target variable.

In yet another aspect, a machine learning server is provided for training a data model to predict data states. The machine learning server is in communication with a first data warehouse system which further includes a warehouse processor and a warehouse memory. The first data warehouse system further includes historical pharmaceutical data associated with one or more pharmaceuticals. The machine learning server includes a processor and a memory. The machine learning server is configured to receive a first portion of historical pharmaceutical data. The first portion includes variables associated with a forecast of a target variable. The machine learning server is configured to apply a deep learning variable importance method to the first portion to identify at least one salient variable. The machine learning server is also configured to apply a combination of a long-short term memory algorithm, a multilayer perceptron algorithm, and a predictive artificial intelligence algorithm to generate a model generation algorithm. The machine learning server is also configured to apply the model generation algorithm to the first portion and the at least one salient variable to generate predictive models for the forecast of the target variable. The machine learning server is also configured to receive a second portion of the historical pharmaceutical data to test the predictive models. The machine learning server is also configured to test the predictive models with the second portion to identify a candidate predictive model that most accurately forecasts the target variable based on the second portion. The machine learning server is also configured to obtain a portion of current pharmaceutical data and apply the portion of current pharmaceutical data to the candidate predictive model to obtain the forecast of the target variable.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4 is a functional block diagram of an example computing device that may be used in the environments described herein.

FIG. 5 is a functional block diagram of a machine learning system for training a data model to predict data states as shown in FIG. 4.

FIG. 6 is a flow diagram representing a method for training a data model to predict data states performed by the machine learning server shown in FIG. 5.

FIG. 7 is a diagram of elements of one or more example computing devices that may be used in the system shown in FIGS. 1-5.

FIG. 8 is a flow diagram of the steps taken to train a data model to predict data states as performed by the machine learning systems described herein.

In the drawings, reference numbers may be reused to identify similar and/or identical elements.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

As used herein, the term “feature selection” refers to the process of selecting a subset of relevant features (e.g., variables or predictors) that are used in the machine learning system to define data models. Feature selection may alternatively be described as variable selection, attribute selection, or variable subset selection. The feature selection process of the machine learning system described herein allows the machine learning server (and related systems) to simplify models to make them easier to interpret, reduce the time to train the systems, reduce overfitting, enhance generalization, and avoid problems in dynamic optimization.

As used herein, the term “hyper-parameter” or “hyperparameter” refers to a parameter whose value is used to control a learning process. By contrast, the values of other parameters (typically node weights) are derived via training. Hyperparameters can be classified as model hyperparameters, that cannot be inferred while fitting the machine to the training set because they refer to the model selection task, or algorithm hyperparameters, that in principle have no influence on the performance of the model but affect the speed and quality of the learning process. An example of a model hyperparameter is the topology and size of a neural network. Examples of algorithm hyperparameters are learning rate and mini-batch size. In one example described herein, hyperparameters may be optimized using a “grid search” or “parameter sweep” entailing searching through a specified subset of the hyperparameter space of a learning algorithm. A grid search algorithm is guided by some performance metric, typically measured by cross-validation on the training set or evaluation on a held-out validation set.

As described herein, a “multilayer perceptron” or “MLP” is a class of feedforward artificial neural network (“ANN”) consisting of at least three layers of nodes: an input layer, a hidden layer and an output layer. Except for the input nodes, each node is a neuron that uses a nonlinear activation function. In most examples, MLP utilizes a supervised learning technique called backpropagation for training. Its multiple layers and non-linear activation distinguish MLP from a linear perceptron. MLP can distinguish data that is not linearly separable.

As described herein, “long short-term memory” or “LSTM” is an artificial recurrent neural network (RNN) architecture used in the field of deep learning. Unlike standard feedforward neural networks, LSTM has feedback connections. It can not only process single data points (such as images), but also entire sequences of data (such as speech or video). For example, LSTM is applicable to tasks such as unsegmented, connected handwriting recognition, speech recognition and anomaly detection in network traffic or IDSs (intrusion detection systems). A common LSTM unit is composed of a cell, an input gate, an output gate and a forget gate. The cell remembers values over arbitrary time intervals and the three gates regulate the flow of information into and out of the cell.

The machine learning systems and methods described herein are configured to address known technological problems confronting computing systems and networks that process data sets, specifically the lack of known effective models between data sets and certain data characteristics. The machine learning systems and methods described are configured to address these known problems particularly as they relate to determining seasonality trends and price forecasts in, for example, pharmaceutical claims processing. As described above, in existing prescription pharmaceutical processing systems, static data models or manual analysis may be used, causing chronic erroneous and improper predictive results. In many examples, the systems can only be improved through the use of manual verification. Yet, even when these steps are taken, forecast data remains error prone and susceptible to future inaccuracy.

The deep learning models and related systems and methods described overcome known deficiencies in previous technological approaches. Using prior approaches, static data models are routinely found to be inaccurate. By contrast, the deep learning models and related machine learning systems and methods provided allow for refactoring and changes to conditions surrounding data sets without requiring manual intervention or verification. As such these deep learning models and related machine learning systems and methods solve technological problems related to forecasting of data (especially seasonal trends and generic price forecasting) that cannot be otherwise resolved using known methods and technologies. In particular, the proposed approach of machine learning using a multi-algorithm approach to automatically build machine learning models in a distributed network, and identify the best model and hyper-parameter tuning, is a significant technological improvement in the technological field of data sciences. Further, the proposed approach allows for active re-factoring to ensure predictive accuracy of time. This approach also allows for re-training of the model and re-tuning using new hyper-parameters. In this manner, the disclosed machine learning methods and systems prevent the data models from becoming static or stale and therefore possibly prone to error.

In the pharmaceutical claims space relevant trend data includes trends in the ratio of forecasted potential dispensed quantities of prescriptions to the prior actual dispensed quantities. This ratio is known as a “factor rate”. A further relevant trend is pricing forecasts based on, in part, average wholesale price (“AWP”) discounts and predicted fill rates.

Therefore, to overcome known problems of forecasting seasonality trends and pricing, a machine learning system is provided. The machine learning system is configured to and capable of defining and utilizing deep learning to create machine learning models. The machine learning system is also configured to perform feature selection from among possible features and determine which feature variables are important to forecast the targets (e.g., seasonality trends and price forecasts). In the example embodiment, the machine learning system includes a machine learning server in communication with a data repository configured to provide historical data sets relevant to the deep learning models and machine learning methods described. The machine learning server includes a processor and a memory. In one example, the data repository is a data warehouse that stores or can otherwise provide historical data sets from pharmaceutical data processing. In another example, the data repository is any database or data store that can provide such historical data sets. The data repository may be, in some examples, directly integrated in pharmaceutical processing systems.

The machine learning server is configured to receive a plurality of data sets associated with each prediction from a data repository that may include a data warehouse, pharmaceutical processing system databases, or an external database or data store. In the example of seasonality trends, the machine learning server receives data including some of the following variables: (a) code number including generic code number (“GCN”); (b) package size; (c) year; (d) month; (e) dispensed quantity for the period; (f) number of claims; and (g) and total average wholesale price (“AWP”) amount. In most examples, (a) and (b) are categorical variables that have pre-defined possible values and (c)-(g) are numeric values that may have suitable possible values. For example, (c) is an integer value for a year and (d) is an integer value for a month while (e)-(g) may be any suitable numeric amount. In general, drug utilization data used to determine seasonal trends is available at a code number (e.g., GCN) level and data is available on a monthly basis. In some examples, drug utilization data may be available based on different groupings and timings. In such examples, the machine learning server may receive data according to the varying groupings and updates. In an example embodiment, the machine learning server forecasts the potential dispensed quantity for an upcoming month as of a given day (e.g., the mid-point of the month or the first or last day of the month) within the month. A predicted “factor rate” may be determined by dividing the potential dispensed quantity with the actual dispensed quantity of the previous month.

In the example of price forecasting, the machine learning server receives data from a data repository that may include a data warehouse, pharmaceutical processing system databases, or an external database or data store. Such data used for price forecasting may include some of the following variables: (a) GCN:Week reflecting a concatenation of values for a generic code number (“GCN”) and a week value reflecting weeks before or after the first generic date (“FGD”); (b) a first generic date (“FGD”) value; (c) a week value reflecting weeks before or after FGD; (d) a WeekDate value reflecting a middle date value for a seven-day period; (e) a generic code number (“GCN”) value; (f) a specific therapeutic class (“STC”) defining the classification of a drug or formulation; (g) a hierarchical ingredient code list (“HICL”) reflecting a name of a drug or formulation; (h) a strength level; (i) a route describing the way that a drug is introduced into a body; (j) a form describing the physical form of a drug (e.g., selected from tablet, capsule, intravenous, cream, suspension, patch, or other forms); (k) a biologic value reflecting whether a drug includes or incorporates genetic material; (l) a name reflecting a commercial name of a drug; (m) a specialty indicator reflecting whether a drug is a specialty; (n) a maintenance indicator reflecting whether a drug is for maintenance; (o) a labelers value indicating the number of manufacturers for a drug; (p) a generic labelers value indicating the number of generic manufacturers for a drug after a patent term ends; (q) a nonauthorized generic labelers value indicating the number of generic manufacturers excluding those that produce an authorized generic; (r) an authorized generic value indicating the number of manufacturers of authorized generics of the drug; (s) a last exclusion week reflecting the last week in which a GCN has less than or equal to one (or <=1) generic labelers; (t) a last nonauthorized generic exclusion week reflecting the last week in which a GCN has less than or equal to one (or <=1) nonauthorized generic labelers; (u) a claim value reflecting the total number of claims for the GCN; (v) a generic claim value reflecting the total number of generic claims for the GCN; (w) a formulary claim value reflecting the total number of formulary claims for the GCN; (x) a mail claim value reflecting the total number of mail claims for the GCN; (y) a quantity value; (z) an average wholesale price (“AWP”) or list price; (aa) a paid ingredient (“PING”) cost typically paid to a pharmacy for a GCN; (ab) a generic fill rate reflecting the total number of generic claims divided by the total number of claims for a GCN; (ac) a formulary fill rate reflecting the total number of formulary claims divided by the total number of claims for a GCN; (ad) a mail fill rate reflecting the total number of mail claims divided by the total number of claims for a GCN; (ae) a paid discount value reflecting a formula of: (1−PING)/(AWP); (af) a market price value reflecting an estimated price that a pharmacy may purchase the GCN given the manufacturer, form, route, and other constraints; and (ag) a market price AWP discount reflecting a calculated percentage discount given by the formula of: (1−market price*quantity/AWP).

In an example embodiment, such data used for price forecasting may be available at a code number (e.g., GCN) level. In at least one example, approximately one year of weekly data may be used to create the data models for price forecasting described. In a further example, fifty-five to sixty weeks of weekly data may be used. The machine learning server is configured to generate models that predict a generic fill rate and an average wholesale price discount (“AWP discount) for the upcoming four weeks. In one example, the generated models (after necessary processing, described below) are used to generate predicted generic fill rates and AWP discounts that are compiled to provide a monthly forecast. The monthly forecast may be provided on any suitable periodic schedule including at the beginning, middle, or end of a month.

In most examples, the machine learning system conducts a series of data pre-processing steps before creating the data models for forecasting. The data pre-processing steps functionally provide automatic cleaning or sanitizing of data by removing or modifying records that may improperly bias the forecasting models. In one example, where certain data variables are expected (based on definitions) to have numeric values, but the actual values are special characters (i.e., reserved characters) or non-numeric values, the pre-processing step deletes the associated records having such values. For example, generic fill rate, AWP discount, and dispensed quantity are expected to have numeric values. If these variables (or other variables defined to have a numeric type) are special or non-numeric values, the associated records are deleted. Similarly, in other cases a non-zero value is expected for certain data variables by definition including, for example, generic fill rate or AWP discount. If the machine learning server determines that values for such variables are zero or null (empty value), in one example it is configured to automatically substitute a previous value such as the previous week data for that variable for the associated record (i.e., the same code number or GCN). Because zero or null values are expected to be non-existent, such reported variables may be determined to be errors and the substitution reduces or removes the risk of the error biasing the model.

In another example, the machine learning server is configured to automatically identify outlier data in the historic data and remove it to avoid biasing the forecasting model. For example, in the example of seasonal trend (or factor rate) forecasting, historic factor rates are calculated based on dividing the current month dispensed quantity by a prior month dispensed quantity (e.g., for the previous month or a prior month) and where the historic factor rate is greater than a predetermined value (e.g., 10), the associated records are deleted because they are likely outliers. In some examples, the predetermined value is determined based on a scan of all historic data to determine statistically likely range of factor rates such that the predetermined value defines the threshold boundaries of factor rates. In a similar manner, the machine learning server is configured to identify outlier values in the historic data for price forecasting by identifying a statistically likely range of values for variables (e.g., features) and removing records with entries outside that range of values.

In another example, the machine learning server applies a data preprocessing step of automatically identifying correlation and features of the variables. Specifically, the machine learning server creates a correlation matrix between each of the variables to identify strongly correlated features that may be salient for predictions and relevant to the deep learning models. This approach is used to provide “automated feature engineering” that allows the machine learning system to adaptively respond to changing data patterns and redefine the underlying data models to respond to the patterns when relevant features change.

In another example, the data preprocessing step entails the machine learning server automatically creating time steps to facilitate the creation of the data models described. As used herein, “time steps” are definitions for data that allow for the processing and analysis of data through specified time intervals. In one example, time steps may be defined using parameters including time intervals, time step repeat intervals, and reference time. A time step interval is the duration of a step (i.e., a period between relevant events). A time step repeat interval is a definition of the frequency of measuring the time step interval. A reference time is a time value used to align time step intervals and time step repeat intervals. Accordingly, in some examples the machine learning server is configured to create time steps for factor rate and price forecasting automatically. In one example, for price forecasting time steps are created for at least the previous four weeks of data. In another example, for factor rate forecasting time steps are created for the previous two years (or twenty-four months) of data.

In the example embodiment, the machine learning server applies an automated feature engineering algorithm to identify salient features from the variables of the historic data. The automated feature engineering algorithm may also be referred to as a deep learning variable importance method that is used to forecast the relative salience (or significance) of variables as predictors to forecast target variables (e.g., factor rates or price forecasts). In one example, the code number (or GCN), package size, year, month, and dispensed quantity have been found to be important (or significant or salient) variables for determining forecasted dispensed quantities and factor rates. In a second example of price forecasting for generics, important (or significant or salient) variables for determining generic price include the following: (a) a week value reflecting weeks before or after FGD; (b) a WeekDate value reflecting a middle date value for a seven-day period; (c) a generic code number (“GCN”) value; (d) a specialty indicator reflecting whether a drug is a specialty; (e) a maintenance indicator reflecting whether a drug is for maintenance; (f) a labelers value indicating the number of manufacturers for a drug; (g) a generic labelers value indicating the number of generic manufacturers for a drug after a patent term ends; (h) a nonauthorized generic labelers value indicating the number of generic manufacturers excluding those that produce an authorized generic; (i) an authorized generic value indicating the number of manufacturers of authorized generics of the drug; (j) a claim value reflecting the total number of claims for the GCN; (k) a generic claim value reflecting the total number of generic claims for the GCN; (l) a formulary claim value reflecting the total number of formulary claims for the GCN; (m) a mail claim value reflecting the total number of mail claims for the GCN; (n) a quantity value; (o) an average wholesale price (“AWP”) or list price; (p) a paid ingredient (“PING”) cost typically paid to a pharmacy for a GCN; (q) a generic fill rate reflecting the total number of generic claims divided by the total number of claims for a GCN; (r) a formulary fill rate reflecting the total number of formulary claims divided by the total number of claims for a GCN; (s) a mail fill rate reflecting the total number of mail claims divided by the total number of claims for a GCN; (t) a paid discount value reflecting a formula of: (1−PING)/(AWP); (u) a market price value reflecting an estimated price that a pharmacy may purchase the GCN given the manufacturer, form, route, and other constraints; and (v) a market price AWP discount reflecting a calculated percentage discount given by the formula of: (1−market price*quantity/AWP).

The machine learning server applies the derived important variables from the deep learning variable importance method to create a plurality of forecasting models including, for example, factor rate models, generic pricing models, AWP discount models, and generic fill rate models. The machine learning server is configured to use historic data and more specifically the derived important variables to forecast future values of factor rates, generic prices, AWP discounts, or generic fill rates. The machine learning server applies a distributed deep learning framework applying algorithms including but not limited to (a) long short-term memory (“LSTM”); (b) multi-layer perceptron (“MLP”); and (c) a predictive artificial intelligence model including but not limited to H20.ai.

The machine learning server is also configured to derive and apply hyperparameters to modify or “tune” the machine learning models. The hyperparameter values and tuning may be accomplished using any suitable method. In one example, possible hyperparameters are determined and configured using grid search. The machine learning server also allows addition, removal, or update of hyperparameters without altering the underlying code.

The machine learning server is configured to build multiple machine learning models for a forecast. The machine learning server tests each of the developed models (for the relevant forecast) and identifies the preferred forecasting model and hyperparameters. In one example, the machine learning server generates and compares approximately seventy-two models and corresponding hyperparameters. The machine learning server performs the comparison by testing the models and corresponding hyperparameters over samples of historic data sets from a relevant period (e.g., the past month). The model and hyperparameter that most accurately provides a relevant forecast is selected as the final model.

The machine learning server is configured to apply the final model and associated hyperparameter to provide a forecast of, for example, the factor rate, AWP discount rate, generic fill rate, or generic price forecast.

Based on the above, the machine learning server provides significant improvements over the known technology in the field. By creating and using the deep learning models described, the machine learning server provides better forecasts for generic prices and factor rates, reduces errors in forecasts, requires less human attention and involvement, improves the reliability of forecasting, and reduces the economic cost of forecasting.

In the example embodiment, the machine learning server employs algorithms and systems including LSTM, MLP, and H20.ai. In some examples, the machine learning server is developed using Scala, Python, Tensorflow, keras, shell scripts, and UNIX.

Generally, the systems and methods described herein are configured to perform at least the following steps: receive a first portion of the plurality of historical pharmaceutical data, wherein the first portion includes variables associated with a forecast of a target variable; apply a deep learning variable importance method to the first portion to identify at least one salient variable; apply a combination of a long-short term memory algorithm, a multilayer perceptron algorithm, and a predictive artificial intelligence algorithm to generate a model generation algorithm; apply the model generation algorithm to the first portion and the at least one salient variable to generate a plurality of predictive models for the forecast of the target variable; receive a second portion of the plurality of historical pharmaceutical data to test the plurality of predictive models; test the plurality of predictive models with the second portion to identify a candidate predictive model that most accurately forecasts the target variable based on the second portion; obtain a portion of current pharmaceutical data; apply the portion of current pharmaceutical data to the candidate predictive model to obtain the forecast of the target variable; apply a grid search to obtain at least one hyperparameter associated with at least one of the plurality of predictive models; test the plurality of predictive models and at least one associated hyperparameter with the second portion to identify a candidate predictive model that most accurately forecasts the target variable based on the second portion; apply the portion of current pharmaceutical data to the candidate predictive model and to the hyperparameter associated with the candidate predictive model to obtain the forecast of the target variable; receive the first portion of the plurality of historical pharmaceutical data, wherein the first portion includes variables associated with a forecast of a target variable representing a generic price forecast; apply a deep learning variable importance method to the first portion to identify at least one salient variable associate with the price forecast; apply the portion of current pharmaceutical data to the candidate predictive model to obtain the price forecast representing a prediction of an average wholesale price and a generic fill rate; receive the first portion of the plurality of historical pharmaceutical data, wherein the first portion includes variables associated with a forecast of a target variable representing a factor rate; apply a deep learning variable importance method to the first portion to identify at least one salient variable associate with the factor rate; apply the portion of current pharmaceutical data to the candidate predictive model to obtain the factor rate forecast; apply at least one pre-processing step to the first portion to obtain a processed first portion; apply a deep learning variable importance method to the processed first portion to identify at least one salient variable; and apply the model generation algorithm to the processed first portion and the at least one salient variable to generate a plurality of predictive models for the forecast of the target variable.

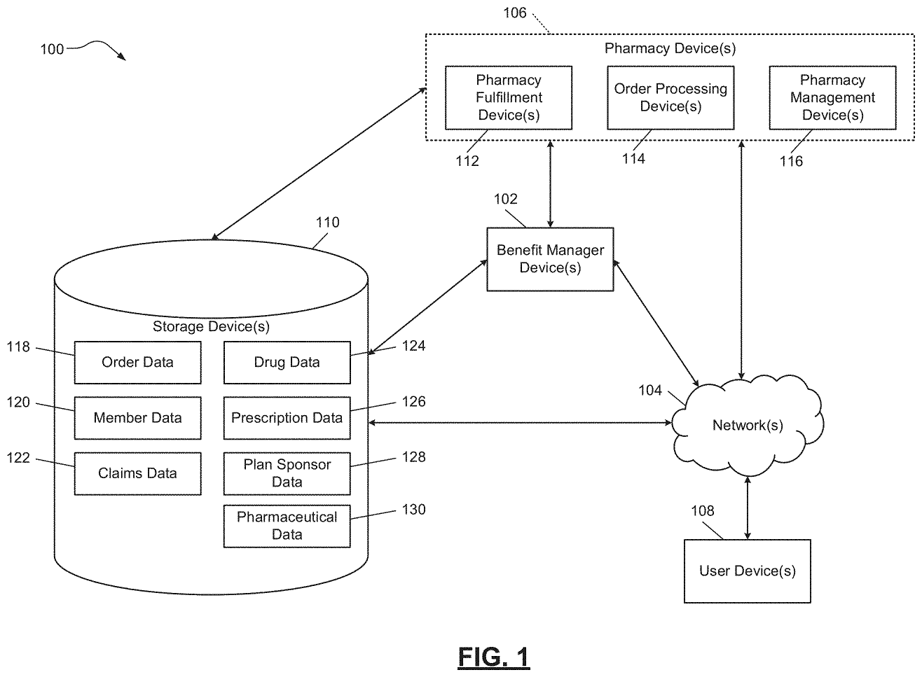

FIG. 1 is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104.

The system 100 may also include one or more user device(s) 108. A user, such as a pharmacist, patient, data analyst, health plan administrator, etc., may access the benefit manager device 102 or the pharmacy device 106 using the user device 108. The user device 108 may be a desktop computer, a laptop computer, a tablet, a smartphone, etc.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in a storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 100) following performance of at least some of the aforementioned operations.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Va.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfilment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, plan sponsor data 128, and/or pharmaceutical data. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the use of the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

The pharmaceutical data 130 includes information regarding particular pharmaceuticals (GCNs). Examples of pharmaceutical data 130 include (a) code number including generic code number (“GCN”); (b) package size; (c) year; (d) month; (e) dispensed quantity for the period; (f) number of claims; and (g) and total average wholesale price (“AWP”) amount. Examples of pharmaceutical data 130 also include: (a) GCN:Week reflecting a concatenation of values for a generic code number (“GCN”) and a week value reflecting weeks before or after the first generic date (“FGD”); (b) a first generic date (“FGD”) value; (c) a week value reflecting weeks before or after FGD; (d) a WeekDate value reflecting a middle date value for a seven-day period; (e) a generic code number (“GCN”) value; (f) a specific therapeutic class (“STC”) defining the classification of a drug or formulation; (g) a hierarchical ingredient code list (“HICL”) reflecting a name of a drug or formulation; (h) a strength level; (i) a route describing the way that a drug is introduced into a body; (j) a form describing the physical form of a drug (e.g., selected from tablet, capsule, intravenous, cream, suspension, patch, or other forms); (k) a biologic value reflecting whether a drug includes or incorporates genetic material; (l) a name reflecting a commercial name of a drug; (m) a specialty indicator reflecting whether a drug is a specialty; (n) a maintenance indicator reflecting whether a drug is for maintenance; (o) a labelers value indicating the number of manufacturers for a drug; (p) a generic labelers value indicating the number of generic manufacturers for a drug after a patent term ends; (q) a nonauthorized generic labelers value indicating the number of generic manufacturers excluding those that produce an authorized generic; (r) an authorized generic value indicating the number of manufacturers of authorized generics of the drug; (s) a last exclusion week reflecting the last week in which a GCN has less than or equal to one (or <=1) generic labelers; (t) a last nonauthorized generic exclusion week reflecting the last week in which a GCN has less than or equal to one (or <=1) nonauthorized generic labelers; (u) a claim value reflecting the total number of claims for the GCN; (v) a generic claim value reflecting the total number of generic claims for the GCN; (w) a formulary claim value reflecting the total number of formulary claims for the GCN; (x) a mail claim value reflecting the total number of mail claims for the GCN; (y) a quantity value; (z) an average wholesale price (“AWP”) or list price; (aa) a paid ingredient (“PING”) cost typically paid to a pharmacy for a GCN; (ab) a generic fill rate reflecting the total number of generic claims divided by the total number of claims for a GCN; (ac) a formulary fill rate reflecting the total number of formulary claims divided by the total number of claims for a GCN; (ad) a mail fill rate reflecting the total number of mail claims divided by the total number of claims for a GCN; (ae) a paid discount value reflecting a formula of: (1−PING)/(AWP); (af) a market price value reflecting an estimated price that a pharmacy may purchase the GCN given the manufacturer, form, route, and other constraints; and (ag) a market price AWP discount reflecting a calculated percentage discount given by the formula of: (1−market price*quantity/AWP).

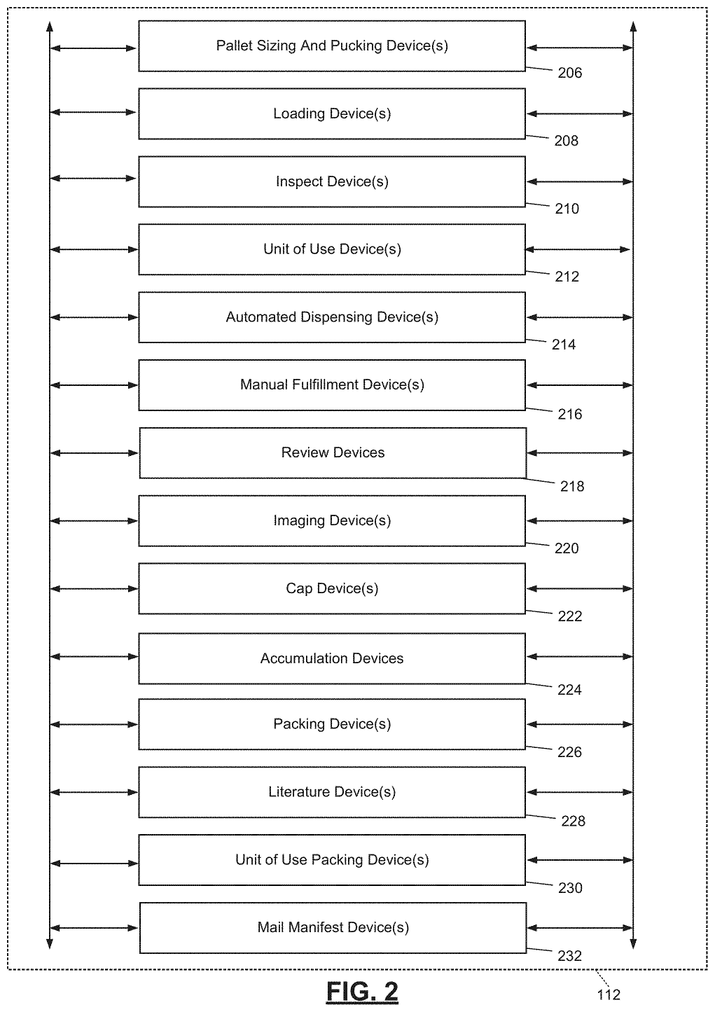

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

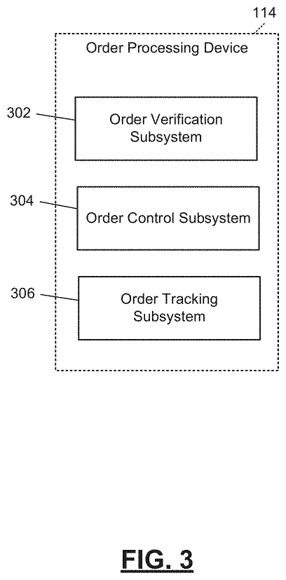

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may include order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

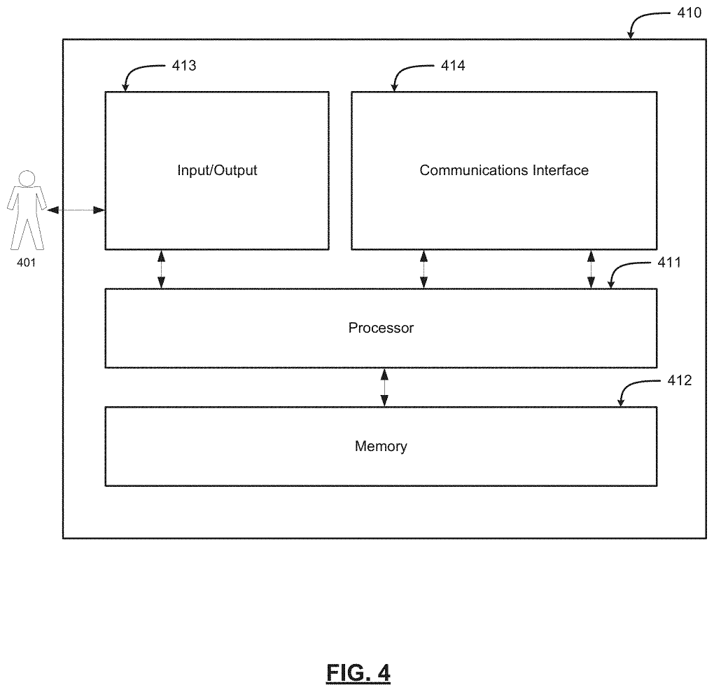

FIG. 4 is a functional block diagram of an example computing device 400 that may be used in the environments described herein. Specifically, computing device 400 illustrates an exemplary configuration of a computing device. Computing device 400 illustrates an exemplary configuration of a computing device operated by a user 401 in accordance with one embodiment of the present disclosure. Computing device 400 may include, but is not limited to, a machine learning server, a data warehouse system, a pharmaceutical data processing system, a host device, an inventory device, and any other system described herein. Computing device 400 may also include pharmacy devices 106 including pharmacy fulfillment devices 112, order processing devices 114, and pharmacy management devices 116, storage devices 110, benefit manager devices 102, and user devices 108 (all shown in FIG. 1), mobile computing devices, stationary computing devices, computing peripheral devices, smart phones, wearable computing devices, medical computing devices, and vehicular computing devices. Alternatively, computing device 400 may be any computing device capable of predicting data states based on pharmaceutical data, including predicting price forecasts, seasonality trends, and other states, as described herein. In some variations, the characteristics of the described components may be more or less advanced, primitive, or non-functional.

In the exemplary embodiment, computing device 400 includes a processor 411 for executing instructions. In some embodiments, executable instructions are stored in a memory area 412. Processor 411 may include one or more processing units, for example, a multi-core configuration. Memory area 412 is any device allowing information such as executable instructions and/or written works to be stored and retrieved. Memory area 412 may include one or more computer readable media.

Computing device 400 also includes at least one input/output component 413 for receiving information from and providing information to user 401. In some examples, input/output component 413 may be of limited functionality or non-functional as in the case of some wearable computing devices. In other examples, input/output component 413 is any component capable of conveying information to or receiving information from user 401. In some embodiments, input/output component 413 includes an output adapter such as a video adapter and/or an audio adapter. Input/output component 413 may alternatively include an output device such as a display device, a liquid crystal display (LCD), organic light emitting diode (OLED) display, or “electronic ink” display, or an audio output device, a speaker or headphones. Input/output component 413 may also include any devices, modules, or structures for receiving input from user 401. Input/output component 413 may therefore include, for example, a keyboard, a pointing device, a mouse, a stylus, a touch sensitive panel, a touch pad, a touch screen, a gyroscope, an accelerometer, a position detector, or an audio input device. A single component such as a touch screen may function as both an output and input device of input/output component 413. Input/output component 413 may further include multiple sub-components for carrying out input and output functions.

Computing device 400 may also include a communications interface 414, which may be communicatively coupleable to a remote device such as a remote computing device, a remote server, or any other suitable system. Communication interface 414 may include, for example, a wired or wireless network adapter or a wireless data transceiver for use with a mobile phone network, Global System for Mobile communications (GSM), 3G, 4G, or other mobile data network or Worldwide Interoperability for Microwave Access (WIMAX). Communications interface 414 is configured to allow computing device 400 to interface with any other computing device or network using an appropriate wireless or wired communications protocol such as, without limitation, BLUETOOTH®, Ethernet, or IEE 802.11. Communications interface 414 allows computing device 400 to communicate with any other computing devices with which it is in communication or connection.

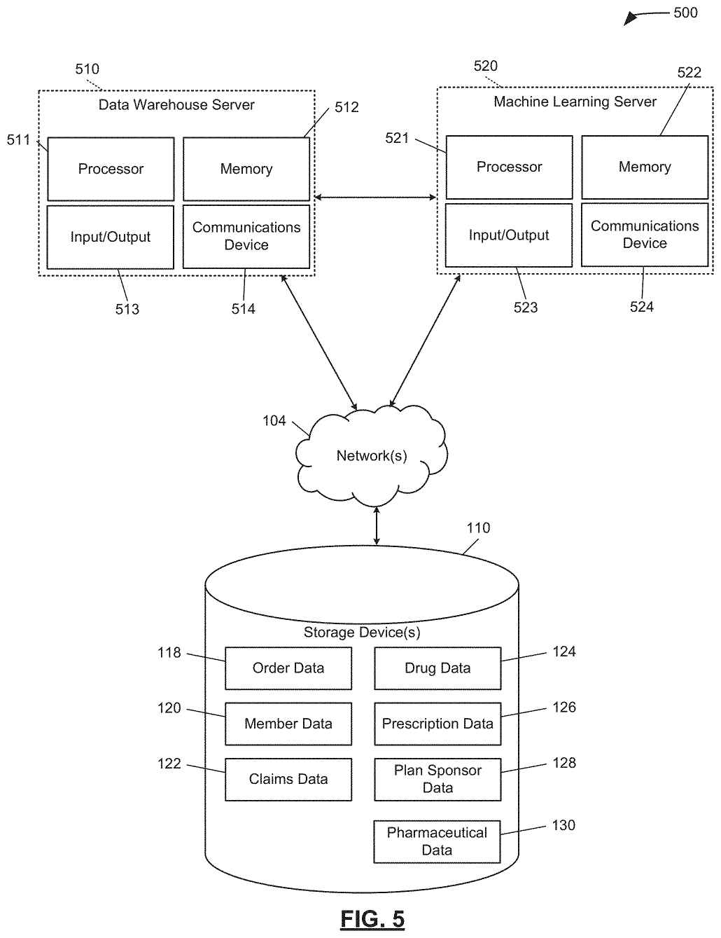

FIG. 5 is a functional block diagram of a machine learning system 500 for training a data model to predict data states including seasonality trends, generic price forecasting, and other price forecasting, including a data warehouse server 510 and a machine learning server 520, which are similar to the computing device 400 shown in FIG. 4. Data warehouse server 510 includes processor 511, memory 512, input/output 513, and communications device 514. Machine learning server 520 includes processor 521, memory 522, input/output 523, and communications device 524. Data warehouse server 510 is in communication with machine learning server 520. Data warehouse server 510 and machine learning server 520 are both in communication with network 104 and capable of accessing storage device 110. As a result, data warehouse server 510 and machine learning server 520 have access to historical and current pharmaceutical data associated with one or more pharmaceuticals (i.e., one or more GCNs) and any associated analytical data available from storage device 110 including order data 118, member data 120, claims data 122, drug data 124, prescription data 126, plan sponsor 128, and/or pharmaceutical data 130. In the example embodiment, data warehouse server 510 has access to historic and current pharmaceutical data described herein through storage device 110, through memory 512, or through other devices available from network 104. Data warehouse server 510 is configured to provide historic and current pharmaceutical data to machine learning server 520 to facilitate the methods described herein. In at least some embodiments, data warehouse server 510 and machine learning server 520 are resident are one computing device that is capable of compiling and integrating historic and current pharmaceutical data along with performing the predictive analytics described.

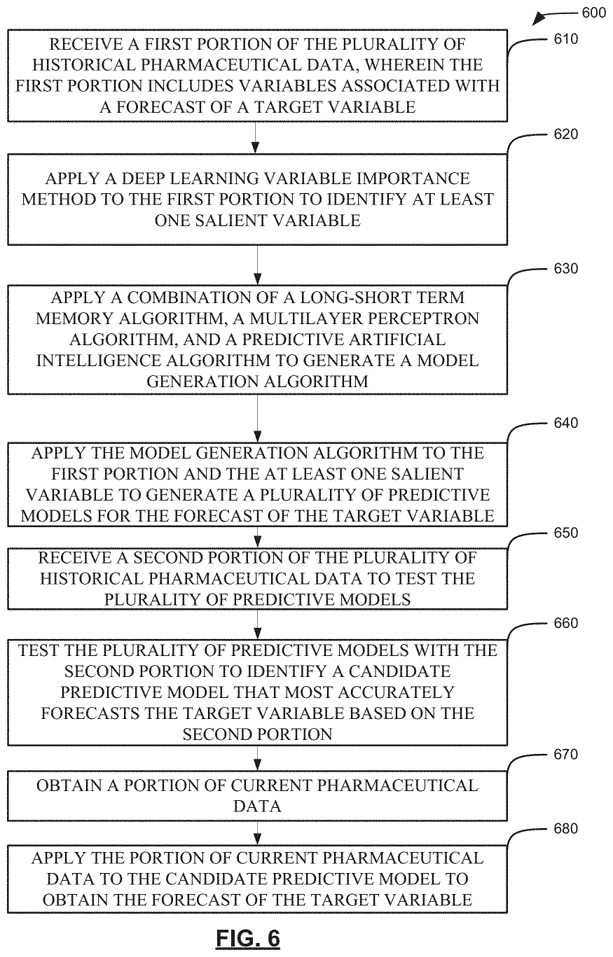

FIG. 6 is a flow diagram representing a method 600 for training a data model to predict data states including seasonality trends, generic price forecasting, and other price forecasting. Method 600 is performed by machine learning server 520 which is configured to receive 610 a first portion of the plurality of historical pharmaceutical data, wherein the first portion includes variables associated with a forecast of a target variable. Machine learning server 520 is also configured to apply 620 a deep learning variable importance method to the first portion to identify at least one salient variable. Machine learning server 520 is also configured to apply 630 a combination of a long-short term memory algorithm, a multilayer perceptron algorithm, and a predictive artificial intelligence algorithm to generate a model generation algorithm. Machine learning server 520 is further configured to apply 640 the model generation algorithm to the first portion and the at least one salient variable to generate a plurality of predictive models for the forecast of the target variable. Machine learning server 520 is also configured to receive 650 a second portion of the plurality of historical pharmaceutical data to test the plurality of predictive models. Machine learning server 520 is configured to test 660 the plurality of predictive models with the second portion to identify a candidate predictive model that most accurately forecasts the target variable based on the second portion. Machine learning server 520 is also configured to obtain 670 a portion of current pharmaceutical data and apply 680 the portion of current pharmaceutical data to the candidate predictive model to obtain the forecast of the target variable.

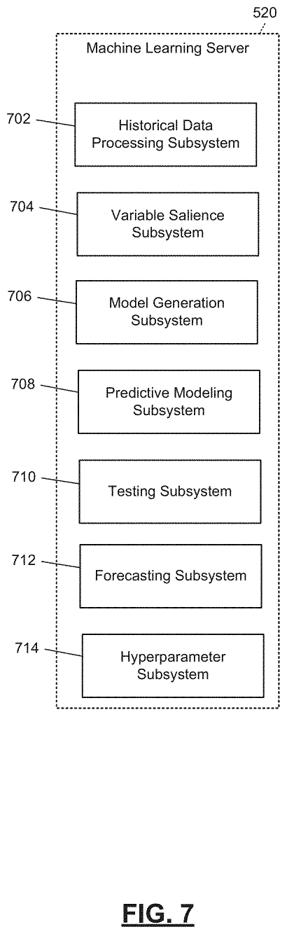

FIG. 7 is a diagram of elements of one or more example computing devices that may be used in the system shown in FIGS. 1-5. As described herein, the elements 702, 704, 706, 708, 710, 712, and 714 are configured to perform the processes and methods described herein. Historical data processing subsystem 702 allows machine learning server 520 to access historical pharmaceutical data and perform any necessary pre-processing steps as described herein to utilize such data. Variable salience subsystem 704 allows machine learning server 520 to apply a deep learning importance method to identify one or more salient features within the historic pharmaceutical data set that are relevant to an associated prediction of, for example, generic price forecast or seasonality trends. In at least some embodiments, application of variable salience subsystem 704 identifies salient features as follows. In one example, the code number (or GCN), package size, year, month, and dispensed quantity have been found to be important (or significant or salient) variables for determining forecasted dispensed quantities and factor rates. In a second example of price forecasting for generics, important (or significant or salient) variables for determining generic price include the following: (a) a week value reflecting weeks before or after FGD; (b) a WeekDate value reflecting a middle date value for a seven-day period; (c) a generic code number (“GCN”) value; (d) a specialty indicator reflecting whether a drug is a specialty; (e) a maintenance indicator reflecting whether a drug is for maintenance; (f) a labelers value indicating the number of manufacturers for a drug; (g) a generic labelers value indicating the number of generic manufacturers for a drug after a patent term ends; (h) a nonauthorized generic labelers value indicating the number of generic manufacturers excluding those that produce an authorized generic; (i) an authorized generic value indicating the number of manufacturers of authorized generics of the drug; (j) a claim value reflecting the total number of claims for the GCN; (k) a generic claim value reflecting the total number of generic claims for the GCN; (l) a formulary claim value reflecting the total number of formulary claims for the GCN; (m) a mail claim value reflecting the total number of mail claims for the GCN; (n) a quantity value; (o) an average wholesale price (“AWP”) or list price; (p) a paid ingredient (“PING”) cost typically paid to a pharmacy for a GCN; (q) a generic fill rate reflecting the total number of generic claims divided by the total number of claims for a GCN; (r) a formulary fill rate reflecting the total number of formulary claims divided by the total number of claims for a GCN; (s) a mail fill rate reflecting the total number of mail claims divided by the total number of claims for a GCN; (t) a paid discount value reflecting a formula of: (1−PING)/(AWP); (u) a market price value reflecting an estimated price that a pharmacy may purchase the GCN given the manufacturer, form, route, and other constraints; and (v) a market price AWP discount reflecting a calculated percentage discount given by the formula of: (1−market price*quantity/AWP). Model generation subsystem 706 allows machine learning server 520 to create necessary models based on a combination of one or more of a long-short term memory algorithm, a multilayer perceptron algorithm, and a predictive artificial intelligence algorithm in order to generate a model generation algorithm. Predictive modeling subsystem 708 allows machine learning server 520 to perform the predictive modeling of applying the model generation algorithm to the first portion and the at least one salient variable to generate a plurality of predictive models for the forecast of the target variable. Testing subsystem 710 allows machine learning server 520 to test the results of each predictive model using a portion of historical pharmaceutical data. Forecasting subsystem 712 allows machine learning server 520 to perform forecasts of relevant data states including, for example, seasonality trends or price forecasts. Hyperparameter subsystem 714 allows machine learning server 520 identify hyperparameters associated with each model based on, for example, grid searching, and apply such hyperparameters in forecasting and testing.

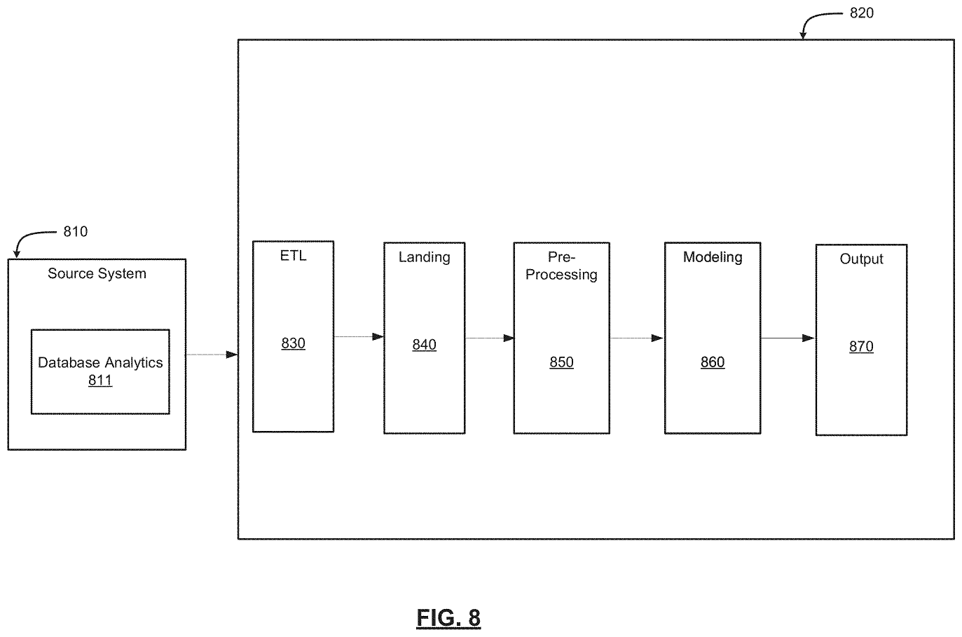

FIG. 8 is a flow diagram of the steps taken to train a data model to predict data states as performed by the machine learning system 500 described herein. Specifically, the diagram reflects a source system 810 that may represent data warehouse server 510 (or similar structures) which provides source pharmaceutical data from a database analytics system 811 to a platform 820 configured to perform the machine learning steps described herein. Platform 820 processes historical (and current) pharmaceutical data through necessary extraction, transformation, and loading (“ETL”) steps 830. In one example, ETL steps 830 are performed using data transformation tools including Sqoop. The historical (and current) pharmaceutical data is also presented to platform 820 using landing steps 840 including, for example, Apache Hadoop Distributed File System (“HDFS”) or Hive. Platform 820 also performs pre-processing steps 840 described herein using necessary tools such as data imputation, data cleansing, and related scripts. Platform 820 also provides modeling algorithm steps 850 to create predictive models as described herein. In the example embodiment, modeling algorithm step 850 includes one or more of long-short term memory algorithm, a multilayer perceptron algorithm, and a predictive artificial intelligence algorithm such as H20.ai. Platform 820 also provides an output step 860 capable of providing forecasts of, for example, seasonality trends or price forecasts. Output step 860 also includes necessary batch processing, file generation, and forecast routing.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave). The term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A machine learning system for training a data model to predict data states, comprising:
a first data warehouse system comprising a warehouse processor and a warehouse memory, the first data warehouse system further including a plurality of historical pharmaceutical data associated with one or more pharmaceuticals;
a machine learning server in communication with the first data warehouse system, the machine learning server comprising a processor and a memory, wherein the machine learning server is configured to:
receive a first portion of the plurality of historical pharmaceutical data, wherein the first portion includes variables associated with a forecast of a target variable;
apply a deep learning variable importance method to the first portion to identify at least one salient variable;
apply a combination of a long-short term memory algorithm, a multilayer perceptron algorithm, and a predictive artificial intelligence algorithm to generate a model generation algorithm;
apply the model generation algorithm to the first portion and the at least one salient variable to generate a plurality of predictive models for the forecast of the target variable;
receive a second portion of the plurality of historical pharmaceutical data to test the plurality of predictive models;
test the plurality of predictive models with the second portion to identify a candidate predictive model that most accurately forecasts the target variable based on the second portion;
obtain a portion of current pharmaceutical data; and
apply the portion of current pharmaceutical data to the candidate predictive model to obtain the forecast of the target variable.

2. The machine learning server of claim 1, wherein the machine learning server is further configured to:
apply a grid search to obtain at least one hyperparameter associated with at least one of the plurality of predictive models.

3. The system of claim 2, wherein the machine learning server is further configured to:
test the plurality of predictive models and at least one associated hyperparameter with the second portion to identify a candidate predictive model that most accurately forecasts the target variable based on the second portion.

4. The system of claim 2, wherein the machine learning server is further configured to:
apply the portion of current pharmaceutical data to the candidate predictive model and to the hyperparameter associated with the candidate predictive model to obtain the forecast of the target variable.

5. The system of claim 1, wherein the machine learning server is further configured to:
receive the first portion of the plurality of historical pharmaceutical data, wherein the first portion includes variables associated with a forecast of a target variable representing a generic price forecast;
apply a deep learning variable importance method to the first portion to identify at least one salient variable associate with the price forecast; and
apply the portion of current pharmaceutical data to the candidate predictive model to obtain the price forecast representing a prediction of an average wholesale price and a generic fill rate.

6. The system of claim 1, wherein the machine learning server is further configured to:
receive the first portion of the plurality of historical pharmaceutical data, wherein the first portion includes variables associated with a forecast of a target variable representing a factor rate;
apply a deep learning variable importance method to the first portion to identify at least one salient variable associate with the factor rate; and
apply the portion of current pharmaceutical data to the candidate predictive model to obtain the factor rate forecast.

7. The system of claim 1, wherein the machine learning server is further configured to:
apply at least one pre-processing step to the first portion to obtain a processed first portion;
apply a deep learning variable importance method to the processed first portion to identify at least one salient variable; and
apply the model generation algorithm to the processed first portion and the at least one salient variable to generate a plurality of predictive models for the forecast of the target variable.

8. A method for training a data model to predict data states performed by a machine learning server in communication with a first data warehouse system including a plurality of historical pharmaceutical data associated with one or more pharmaceuticals, the machine learning server including a processor and a memory, said method comprising:
receiving a first portion of the plurality of historical pharmaceutical data, wherein the first portion includes variables associated with a forecast of a target variable;
applying a deep learning variable importance method to the first portion to identify at least one salient variable;
applying a combination of a long-short term memory algorithm, a multilayer perceptron algorithm, and a predictive artificial intelligence algorithm to generate a model generation algorithm;
applying the model generation algorithm to the first portion and the at least one salient variable to generate a plurality of predictive models for the forecast of the target variable;
receiving a second portion of the plurality of historical pharmaceutical data to test the plurality of predictive models;
testing the plurality of predictive models with the second portion to identify a candidate predictive model that most accurately forecasts the target variable based on the second portion;
obtaining a portion of current pharmaceutical data; and
applying the portion of current pharmaceutical data to the candidate predictive model to obtain the forecast of the target variable.

9. The method of claim 8, further comprising:
applying a grid search to obtain at least one hyperparameter associated with at least one of the plurality of predictive models.

10. The method of claim 9, further comprising:
testing the plurality of predictive models and at least one associated hyperparameter with the second portion to identify a candidate predictive model that most accurately forecasts the target variable based on the second portion.

11. The method of claim 9, further comprising:
applying the portion of current pharmaceutical data to the candidate predictive model and to the hyperparameter associated with the candidate predictive model to obtain the forecast of the target variable.

12. The method of claim 11, further comprising:
receiving the first portion of the plurality of historical pharmaceutical data, wherein the first portion includes variables associated with a forecast of a target variable representing a generic price forecast;
applying a deep learning variable importance method to the first portion to identify at least one salient variable associate with the price forecast; and
applying the portion of current pharmaceutical data to the candidate predictive model to obtain the price forecast representing a prediction of an average wholesale price and a generic fill rate.

13. The method of claim 8, further comprising:
receiving the first portion of the plurality of historical pharmaceutical data, wherein the first portion includes variables associated with a forecast of a target variable representing a factor rate;
applying a deep learning variable importance method to the first portion to identify at least one salient variable associate with the factor rate; and
applying the portion of current pharmaceutical data to the candidate predictive model to obtain the factor rate forecast.

14. The method of claim 8, further comprising:
applying at least one pre-processing step to the first portion to obtain a processed first portion;
applying a deep learning variable importance method to the processed first portion to identify at least one salient variable; and
applying the model generation algorithm to the processed first portion and the at least one salient variable to generate a plurality of predictive models for the forecast of the target variable.

15. A machine learning server for training a data model to predict data states, the machine learning server in communication with a first data warehouse system further including a warehouse processor and a warehouse memory, the first data warehouse system including a plurality of historical pharmaceutical data associated with one or more pharmaceuticals, said machine learning server comprising a processor and a memory, wherein said processor is configured to:
receive a first portion of the plurality of historical pharmaceutical data, wherein the first portion includes variables associated with a forecast of a target variable;
apply a deep learning variable importance method to the first portion to identify at least one salient variable;
apply a combination of a long-short term memory algorithm, a multilayer perceptron algorithm, and a predictive artificial intelligence algorithm to generate a model generation algorithm;
apply the model generation algorithm to the first portion and the at least one salient variable to generate a plurality of predictive models for the forecast of the target variable;
receive a second portion of the plurality of historical pharmaceutical data to test the plurality of predictive models;
test the plurality of predictive models with the second portion to identify a candidate predictive model that most accurately forecasts the target variable based on the second portion;
obtain a portion of current pharmaceutical data; and
apply the portion of current pharmaceutical data to the candidate predictive model to obtain the forecast of the target variable.

16. The machine learning server of claim 15, further configured to:
apply a grid search to obtain at least one hyperparameter associated with at least one of the plurality of predictive models.

17. The machine learning server of claim 16, further configured to:
test the plurality of predictive models and at least one associated hyperparameter with the second portion to identify a candidate predictive model that most accurately forecasts the target variable based on the second portion.

18. The machine learning server of claim 16, further configured to:
apply the portion of current pharmaceutical data to the candidate predictive model and to the hyperparameter associated with the candidate predictive model to obtain the forecast of the target variable.

19. The machine learning server of claim 15, further configured to:
receive the first portion of the plurality of historical pharmaceutical data, wherein the first portion includes variables associated with a forecast of a target variable representing a generic price forecast;
apply a deep learning variable importance method to the first portion to identify at least one salient variable associate with the price forecast; and
apply the portion of current pharmaceutical data to the candidate predictive model to obtain the price forecast representing a prediction of an average wholesale price and a generic fill rate.

20. The machine learning server of claim 15, further configured to:
receive the first portion of the plurality of historical pharmaceutical data, wherein the first portion includes variables associated with a forecast of a target variable representing a factor rate;
apply a deep learning variable importance method to the first portion to identify at least one salient variable associate with the factor rate; and
apply the portion of current pharmaceutical data to the candidate predictive model to obtain the factor rate forecast.
